(19) United States
(12) Patent Application Publication
(10) Pub. No.: US 2026/0207116 A1
(43) Pub. Date: Jul. 23, 2026

(54) MAGNETIC-STIMULATION COIL

(71) Applicant: QUANTALX NEUROSCIENCE LTD, Kfar Saba (IL)

(72) Inventors: Hilla Fogel, Mazor (IL); Iftach Dolev, Zur Moshe (IL); Eyal Rotman, Haifa (IL); Boris Ashkinezer, Akko (IL)

(21) Appl. No.: 19/138,272

(22) PCT Filed: Dec. 22, 2023
(86) PCT No.: PCT/IB2023/063172

§ 371 Date: Jun. 12, 2025

Related U.S. Application Data
(60) Provisional application No. 63/435,011, filed on Dec. 23, 2022.

(51) Int. Cl. A61B 5/377; A61B 5/00

(52) U.S. Cl. CPC A61B 5/377; A61B 5/4088; A61B 5/6803

(57) ABSTRACT

An apparatus includes a magnetic-stimulation device (20) including a magnetic-stimulation coil (21) that is formed from a flattened wire (33) that is wound such as to form two circles (35) that are separated from each other, a ratio between a width (W1) and a thickness of the flattened wire being greater than 3:1. The magnetic-stimulation device further includes a housing (38) that houses the magnetic-stimulation coil. Other embodiments are also described.

### CROSS-REFERENCE TO RELATED APPLICATIONS

The present application claims priority to U.S. Provisional Application 63/435,011 to Fogel et al., entitled “Magnetic-stimulation coil,” filed Dec. 23, 2022, whose disclosure is incorporated herein by reference.

### FIELD OF EMBODIMENTS OF THE INVENTION

The present invention relates to methods and apparatus for use in medical procedures, and particularly apparatus and methods for applying magnetic stimulation.

### BACKGROUND

In the neuropsychiatric world, the ability to deal with the expanding risk of age-associated brain disorders, such as Alzheimer's disease (AD), and other neurodegenerative-psychiatric disorders, is limited by the tools that are available for the evaluation and monitoring of brain health status. For example, though technologies such as magnetic resonance imaging (MRI) and computed tomography (CT) provide high-resolution images of the structural topology of the brain's neural network, these technologies lack the ability to directly monitor brain functionality. Functional MRI (fMRI) and positron emission tomography (PET-CT) are used for indirect measurements, such as measurements of blood flow, which correlate with regional changes in levels of brain activity, but cannot be used for the direct evaluation of the logical topology of the network. Moreover, these tools may not provide any valuable insights when evaluating brain health during normal aging or age-related pathological deterioration.

Electrophysiological measurements have been used extensively to characterize and monitor brain network activity over the last seven decades. Electrophysiological measurements can be generally divided into two groups of parameters: network integrity, i.e., the connectivity and coherence of the network, and network plasticity, also referred to as “neuroplasticity” or “brain plasticity.” Network connectivity depends on the synchronous activation of neurons. Network coherence refers to the level of synchrony between two or more brain regions and is used to determine the strength of connectivity between specific brain regions. Neuroplasticity is an ability of the brain to continuously adapt its functional and structural organization to changing requirements. Neuronal plasticity allows the brain to reorganize neuronal networks in response to environmental stimulation, to remember information, and to recover from brain and spinal-cord injuries. Neuronal plasticity is essential to the establishment and maintenance of brain circuitry.

Magnetic stimulation is a non-invasive brain stimulation method that allows the study of human cortical function in vivo. Using magnetic stimulation for examining human cortical functionality is enhanced by combining such stimulation with registration of an electrical evoked response, such as an electroencephalograph (EEG). EEG provides an opportunity to directly measure the cerebral response to magnetic stimulation, measuring the cortical evoked potential. An important feature of the evoked potential topography is that even though only one cortical hemisphere is stimulated, bi-hemispheric EEG responses are evoked with different features. Magnetic-stimulation-evoked activity propagates from the stimulation site ipsilaterally via association fibers, contralaterally via transcallosal fibers, and to subcortical structures via projection fibers. A single stimulating pulse delivered over the primary motor cortex (M1) results in a sequence of positive and negative EEG peaks at specific latencies (typically, negative peaks at 45 ms (N45) and 100 ms (N100) after stimulation, and positive peaks at 60 ms (P60) and 180 ms (P180) after stimulation). This pattern of response indicates synaptic activity. These evoked cortical potentials last for up to 300 ms both in the vicinity of the stimulation and in remote interconnected brain areas.

### SUMMARY OF EMBODIMENTS

In accordance with some applications of the present invention, a diagnostic procedure is performed on a patient in which a magnetic-stimulation device magnetically stimulates the patient's brain. Typically, the magnetic-stimulation device includes one or more magnetic-stimulation coils, which are configured to generate a magnetic field. Typically, the electrical response of the patient's brain that is evoked by the magnetic stimulation is detected using an electrical signal detector, such as an electroencephalograph (EEG) detector. Typically, the electrical signal detector includes a plurality of electrodes that are configured to be placed in contact with the patient's head. Typically, a computer processor drives the magnetic-stimulation device to magnetically stimulate the patient's brain and receives a detected magnetic-stimulation-evoked signal from the electrical signal detector. Based upon one or more parameters of the detected signal, the computer processor performs a diagnosis of the patient. Typically, the computer processor outputs the results of the diagnosis on an output device, such as a display. For some applications, the computer processor determines that the patient is suspected of suffering from a degenerative disorder such as Parkinson's disease, vascular dementia, Alzheimer's disease, or frontotemporal dementia. For some applications, the computer processor performs a differential diagnosis based upon one or more parameters of the detected signal.

For some applications, the plurality of electrodes, which are configured to record the electrical response of the brain to the stimulation, are arranged within a cap that is configured for wearing on the patient's head. For example, the cap may be shaped to define multiple electrode-holding orifices shaped to hold the electrodes. Alternatively or additionally, the magnetic-stimulation device is configured for reversible coupling to the cap. Typically, this reversible coupling maintains the position of the magnetic-stimulation device relative to the patient's head even in the event that the patient moves their head.

For example, in some embodiments, the cap comprises a piece of material and multiple coupling pads coupled to the piece of material. The magnetic-stimulation device comprises a coil housing and one or more (e.g., four) straps coupled to the coil housing, each of the straps being configured to couple to any one of the coupling pads (e.g., via a hook-and-loop fastener) so as to couple the coil housing to the cap while the cap is worn on the head of the patient. The magnetic-stimulation device further comprises a coil housed within the coil housing and configured to magnetically stimulate the patient's brain while the coil housing is coupled to the cap.

Typically, the magnetic-stimulation coil comprises a flattened wire (i.e., a wire shaped as a band or a strip), which is wound into two circles that are separated from each other. For some applications, the width of the flattened wire is more than 4 mm (e.g., more than 5 mm) and/or less than 7 mm (e.g., less than 6 mm), for example, 4 mm-7 mm, or 5 mm-6 mm. Alternatively or additionally, the thickness of the flattened wire is more than 0.8 mm (e.g., more than 1 mm) and/or less than 1.6 mm (e.g., less than 1.4 mm), for example, 0.8 mm-1.6 mm, or 1 mm-1.4 mm. Alternatively or additionally, the ratio between the width of the flattened wire and the thickness of each the flattened wire is more than 3:1 (e.g., more than 4:1) and/or less than 6:1 (e.g., less than 5:1), for example between 3:1 and 6:1 or between 4:1 and 5:1.

For some applications, each of the circles includes between 6 and 10 complete turns of the flattened wire, for example, 8 complete turns of the flattened wire. For other applications, each of the circles includes between 10 and 16 complete turns of the flattened wire, for example, 12-14 complete turns of the flattened wire. For some applications, the circles that are formed by the flattened wire are separated from each other (i.e., the outer edges of each of the circles are separated from each other) by more than 10 mm (e.g., more than 12 mm) and/or less than 18 mm (e.g., less than 16 mm), for example, 10 mm-18 mm or 12 mm-16 mm.

Advantageously, as further described below with reference to FIG. 5A, a coil having parameters as described herein allows the magnetic-stimulation device to have a relatively low weight and/or volume relative to conventional coils. This allows the magnetic-stimulation device to be readily and comfortably positioned (and repositioned, if required) over the cap, e.g., by reversible coupling to the cap as described above. Moreover, the coil can provide a sufficiently-strong magnetic field at the desired depth within the patient's brain without overheating and without producing an overly-strong magnetic field at the patient's scalp, which could cause the patient discomfort.

Typically, the magnetic-stimulation coil is fully (and typically hermetically) encapsulated by a cover, also referred to below as a “housing.” For some applications, the cover comprises two pieces of plastic, an underside and an upper side, that are welded together. In some embodiments, the cover also contains one or more printed circuit boards (PCBs) comprising circuitry for facilitating the stimulation, such as one or more light emitting diodes (LEDs) and/or temperature sensors. One or more filters protect the circuitry from the significant common-mode voltage generated by the coil.

There is therefore provided, in accordance with some embodiments of the present invention, an apparatus including a magnetic-stimulation device. The magnetic-stimulation device includes a magnetic-stimulation coil that is formed from a flattened wire that is wound such as to form two circles that are separated from each other, a ratio between a width and a thickness of the flattened wire being greater than 3:1. The magnetic-stimulation device further includes a housing that houses the magnetic-stimulation coil.

In some embodiments, an inner diameter of each of the circles is between 21 and 31 mm.

In some embodiments, the inner diameter is between 24 and 28 mm.

In some embodiments, the flattened wire is wound such that each of the two circles includes between 6 and 10 complete turns of the flattened wire.

In some embodiments, the flattened wire is wound such that each of the two circles includes between 10 and 16 complete turns of the flattened wire.

In some embodiments, the housing hermetically seals the magnetic-stimulation coil.

In some embodiments, the housing includes a compartment and a cover that are welded to each other.

In some embodiments, the ratio between the width and the thickness of the flattened wire is between 3:1 and 6:1.

In some embodiments, the ratio between the width and the thickness of the flattened wire is greater than 4:1.

In some embodiments, the width of the flattened wire is more than 4 mm.

In some embodiments, the width of the flattened wire is more than 5 mm.

In some embodiments, the thickness of the flattened wire is less than 1.6 mm.

In some embodiments, the thickness of the flattened wire is less than 1.4 mm.

In some embodiments, the two circles are separated from each other by more than 10 mm.

In some embodiments, the two circles are separated from each other by more than 12 mm.

In some embodiments, the two circles are separated from each other by between 10 mm and 18 mm.

In some embodiments,
the magnetic-stimulation coil is configured to generate a magnetic field having a magnetic-field strength, and
a ratio of the magnetic-field strength at a 2 cm distance from a surface of the coil along a central axis of the coil to the magnetic-field strength at the surface of the coil is greater than 2:3.

In some embodiments, the ratio of the magnetic-field strength at the 2 cm distance from the surface of the coil along the central axis of the coil to the magnetic-field strength at the surface of the coil is greater than 1:1.

There is further provided, in accordance with some embodiments of the present invention, a system, including:
the apparatus;
a plurality of electrodes; and
at least one computer processor configured to:
   drive the magnetic-stimulation device to apply a magnetic stimulation to a brain of the patient via the magnetic-stimulation coil,
   receive a magnetic-stimulation-evoked signal that is detected by the electrodes, and
   diagnose the patient, by analyzing the magnetic-stimulation-evoked signal.

In some embodiments, the at least one computer processor is configured to drive the magnetic-stimulation device to apply the magnetic stimulation to a depth of at least 2 cm from a scalp of the patient.

In some embodiments, the magnetic-stimulation coil is configured not to overheat when the magnetic stimulation is applied to the depth of at least 2 cm.

In some embodiments, the magnetic-stimulation coil is configured not to cause discomfort by heating a scalp of the patient when the magnetic stimulation is applied to the depth of at least 2 cm.

In some embodiments, the plurality of electrodes are arranged within a cap that is configured to be placed on a head of the patient.

In some embodiments, the magnetic-stimulation device is configured to be reversibly coupled to the cap.

There is further provided, in accordance with some embodiments of the present invention, an apparatus including a coil, configured to magnetically stimulate a brain of a patient during a stimulation procedure, and a printed circuit board. The printed circuit board includes circuitry, configured to produce an output for facilitating the stimulation procedure, and at least one filtering wire, configured to protect the circuitry by filtering a common-mode voltage from the coil. The apparatus further includes a housing that houses the coil and the printed circuit board.

In some embodiments, the housing hermetically seals the coil.

In some embodiments, the housing includes a compartment and a cover that are welded to each other.

In some embodiments, the circuitry includes one or more light emitting diodes configured to emit light indicating a stage of the stimulation procedure.

In some embodiments, the circuitry includes one or more temperature sensors configured to output signals indicating a temperature within the housing.

There is further provided, in accordance with some embodiments of the present invention, a system including a cap configured for wearing on a head of a patient, the cap including a piece of material and multiple coupling pads coupled to the piece of material. The system further includes a magnetic-stimulation device including a coil housing, one or more straps coupled to the coil housing, each of the straps being configured to couple to any one of the coupling pads so as to couple the coil housing to the cap while the cap is worn on the head of the patient, and a coil housed within the coil housing and configured to magnetically stimulate a brain of the patient while the coil housing is coupled to the cap.

In some embodiments, the cap is shaped to define multiple electrode-holding orifices shaped to hold respective electrodes configured to record an electrical response of the brain to the stimulation.

In some embodiments, the coupling pads include loops, and the straps include hooks configured to couple to the loops.

In some embodiments, the coupling pads include hooks, and the straps include loops configured to couple to the hooks.

In some embodiments, the magnetic-stimulation device includes four straps.

In some embodiments, the magnetic-stimulation device further includes a button, and the magnetic-stimulation device is configured to emit a test electromagnetic pulse in response to a pushing of the button.

In some embodiments, a mass of the coil housing, with the coil, is less than 500 g.

In some embodiments, the coupling pads are positioned on the piece of material such that, when the cap is worn on the head of the patient, the coil housing is couplable to the cap over a frontal cortex of the brain of the patient.

In some embodiments, the coupling pads are positioned on the piece of material such that, when the cap is worn on the head of the patient, the coil housing is couplable to the cap over a primary motor cortex of the brain of the patient.

In some embodiments, the coupling pads are positioned on the piece of material such that, when the cap is worn on the head of the patient, the coil housing is couplable to the cap over a dorsolateral prefrontal cortex of the brain of the patient.

In some embodiments, the coupling pads are positioned on the piece of material such that, when the cap is worn on the head of the patient, the coil housing is couplable to the cap over a parietal cortex of the brain of the patient.

In some embodiments, the coupling pads are positioned on the piece of material such that, when the cap is worn on the head of the patient, the coil housing is couplable to the cap over an occipital cortex of the brain of the patient.

In some embodiments, the coupling pads are positioned on the piece of material such that, when the cap is worn on the head of the patient, the coil housing is couplable to the cap over a temporal cortex of the brain of the patient.

In some embodiments, the material is stretchable.

In some embodiments, the material includes synthetic rubber.

In some embodiments, the material includes spandex.

There is further provided, in accordance with some embodiments of the present invention, an apparatus including a coaxial cable. The coaxial cable includes an inner conductor, an outer conductor, which is coaxial with the inner conductor, an inner jacket, which insulates the inner conductor and outer conductor from one another, and an outer jacket, which insulates the outer conductor from a surrounding environment and has an outer diameter less than 1.4 cm. A mass of the coaxial cable is less than 700 g, and a bend radius of the coaxial cable is less than five times the outer diameter.

In some embodiments, the coaxial cable further includes:
one or more insulated control wires passing through the inner conductor; and
another inner jacket, which insulates the control wires from the inner conductor.

The present invention will be more fully understood from the following detailed description of embodiments thereof, taken together with the drawings, in which:

### BRIEF DESCRIPTION OF THE DRAWINGS

FIG. 1 is a schematic illustration of a clinician performing a diagnostic procedure on a patient using a diagnostic system, in accordance with some embodiments of the present invention;

FIG. 2A is a schematic illustration of a control unit with a magnetic-stimulation device, in accordance with some embodiments of the present invention;

FIG. 2B is a schematic illustration of a magnetic-stimulation device connected to a cable, in accordance with some embodiments of the present invention;

FIG. 3 is a schematic illustration of a cross-sectional view of a cable, in accordance with some embodiments of the present invention;

FIG. 4 is a schematic illustration of a cap, in accordance with some embodiments of the present invention;

FIG. 5A is a schematic illustration of a coil housing, in accordance with some embodiments of the present invention;

FIG. 5B is a schematic illustration of a compartment of a coil housing, in accordance with some embodiments of the present invention; and

FIG. 6 shows results of a simulated magnetic stimulation using a coil having parameters in accordance with some embodiments of the present invention.

### DETAILED DESCRIPTION

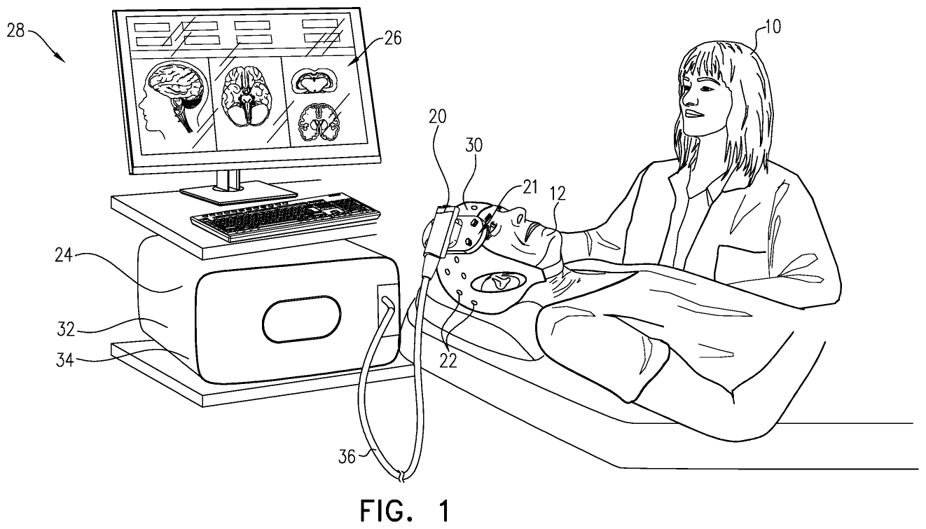

Reference is initially made to FIG. 1, which is a schematic illustration of a clinician 10 performing a diagnostic procedure on a patient 12 using a diagnostic system 28, in accordance with some embodiments of the present invention. The diagnostic procedure may be performed, for example, if patient 12 is suspected of suffering from a neurodegenerative disorder.

System 28 comprises a magnetic-stimulation device 20 configured for placement near the head of patient 12, e.g., by virtue of being placed over (e.g., coupled to) a cap 30 worn over the head. Device 20 comprises at least one coil configured to generate a magnetic field, which stimulates activity within the brain of patient 12. In general, device 20 may be placed over any suitable portion of the frontal cortex (e.g., the primary motor cortex or dorsolateral prefrontal cortex), occipital cortex, parietal cortex, or temporal cortex of the patient's brain, so as to stimulate that portion of the brain.

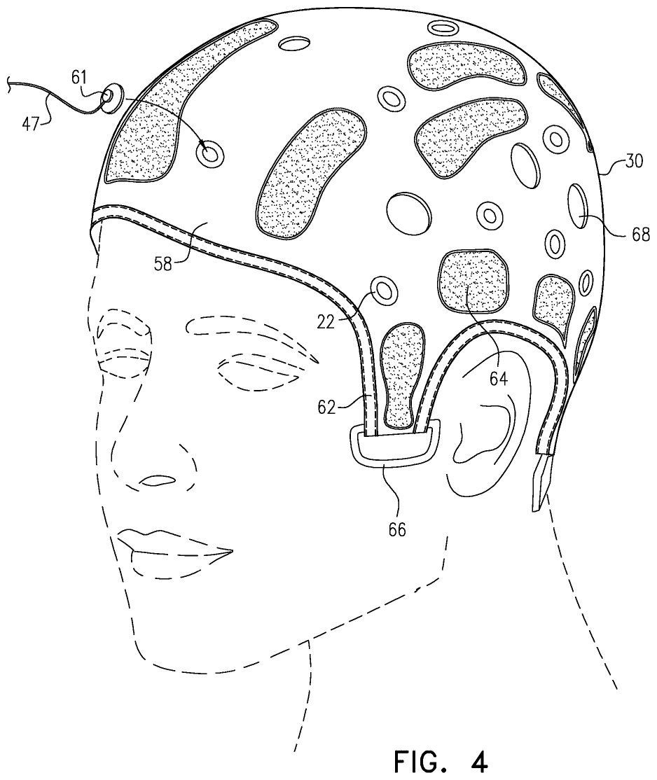

System 28 further comprises multiple electrodes 61 (FIG. 4) configured to record the signals produced by the brain in response to the stimulation. Electrodes 61 may be coupled to the patient's head via a low-impedance adhesive material. Alternatively, the electrodes may be coupled to cap 30 (e.g., via electrode-holding orifices 22 in the cap) such that, when cap 30 is fittingly placed over the patient's head, the electrodes contact the head.

System 28 further comprises a control unit 24 comprising a signal generator 34 and other circuitry, such as analog-to-digital (A/D) conversion circuitry and/or denoising circuitry. Typically, control unit 24 is connected to device 20 via a cable 36. A computer processor 32 which may belong to control unit 24 or to an external device, such as a laptop, in communication with control unit 24 (e.g., via a universal serial bus (USB) cable)—is configured to drive signal generator 34 to generate electrical signals. These signals flow through device 20 via cable 36, thus causing the device to generate a magnetic field, which in turn evokes signals (or “potentials”) in the patient's brain. These signals are recorded by the electrodes and passed from the electrodes, via respective leads 47 (FIG. 4) or via wireless transfer, to the control unit. After optional denoising and digitization within control unit 24, processor 32 receives the signals.

Processor 32 is further configured to process the signals so as to perform a diagnosis, such as a differential diagnosis, with respect to a condition such as Parkinson's disease, vascular dementia, Alzheimer's disease, or frontotemporal dementia. The processor additionally outputs an output indicating the diagnosis; for example, the processor may display the output on a display 26.

Typically, in processing the signals, the processor computes one or more measures of neurophysiological activity exhibited in the signals. For example, the processor may compare the measures to respective thresholds or input the measures to a model, such as a neural network or logistic regression model, which is calibrated to output a diagnosis.

In some embodiments, the computed measures of neurophysiological activity include a waveform adherence measure, which quantifies the similarity between the waveform of a portion of the signals and the waveform of a corresponding portion of a benchmark signal, which may be obtained, for example, from relevant literature. Typically, the benchmark signal represents the response of a normal subject.

One such waveform adherence measure is a wide waveform adherence measure, which quantifies the similarity over a relatively long (or “wide”) portion of the signals. For example, the portion may begin 15-55 ms from the start of the signals (i.e., from the end of the stimulation) and have a duration of 300-350 ms.

Another such waveform adherence measure is a late waveform adherence measure, which quantifies the similarity over a relatively late portion of the signals. For example, the portion may begin at least 60 ms (e.g., at least 80 ms) from the start of the signals. The duration of the portion may be, for example, between 120 and 160 ms.

Yet another such waveform adherence measure is an early waveform adherence measure, which quantifies the similarity over a relatively early portion of the signals. For example, the portion may begin less than 50 ms from the start of the signals. The duration of the portion may be, for example, between 120 and 160 ms. As a specific example, the portion may begin 35 ms post-stimulus and have a duration of 145 ms.

Alternatively or additionally, the computed measures of neurophysiological activity include a cortical excitability measure, which is based on an amplitude of a portion of the signals. For example, the cortical excitability measure may be based on an integral of the signals (which depends on the amplitude) or on a statistic of the amplitude, such as the mean average deviation of the amplitude.

Alternatively or additionally, the computed measures of neurophysiological activity include a waveform excitability measure for a portion of signals, which is based both on the amplitude of the portion of the signals and on the similarity of the waveform to a benchmark waveform.

Alternatively or additionally, the computed measures of neurophysiological activity include an interhemispheric connectivity measure, which quantifies the similarity between the response of the right side of the patient's brain to the stimulation and the response of the left side of the patient's brain to the stimulation.

Alternatively or additionally, the computed measures of neurophysiological activity include a main-peak latency measure, which quantifies the latency of a main peak in the signals, the slope of a line passing through two main peaks, and/or a difference between two latencies.

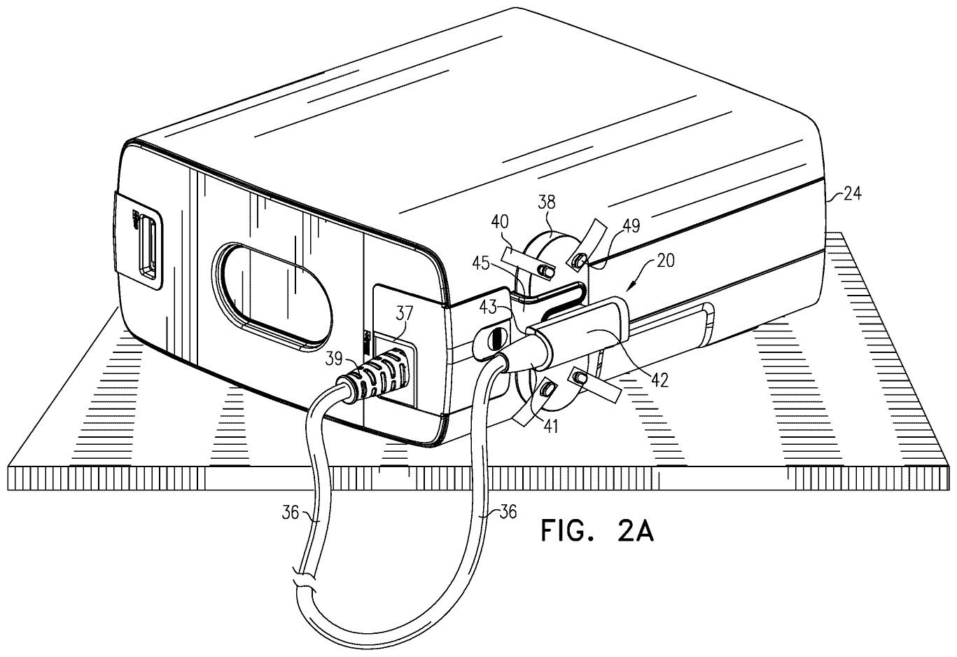

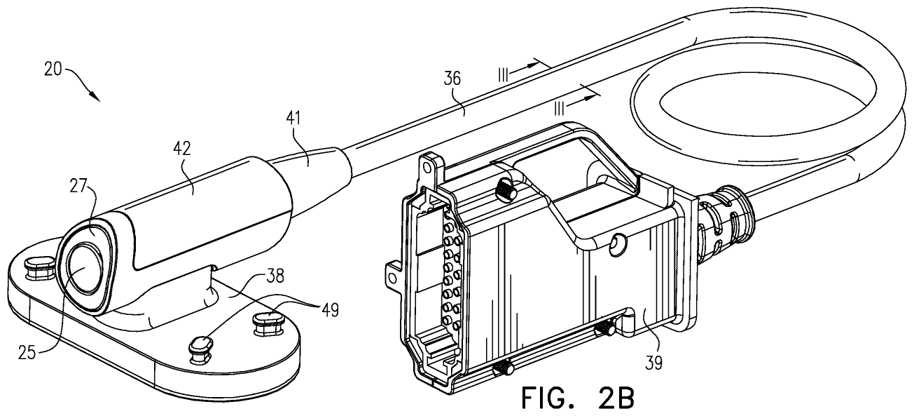

Reference is now made to FIG. 2A, which is a schematic illustration of control unit 24 with magnetic-stimulation device 20, in accordance with some embodiments of the present invention. Reference is additionally made to FIG. 2B, which is a schematic illustration of magnetic-stimulation device 20 connected to cable 36, in accordance with some embodiments of the present invention.

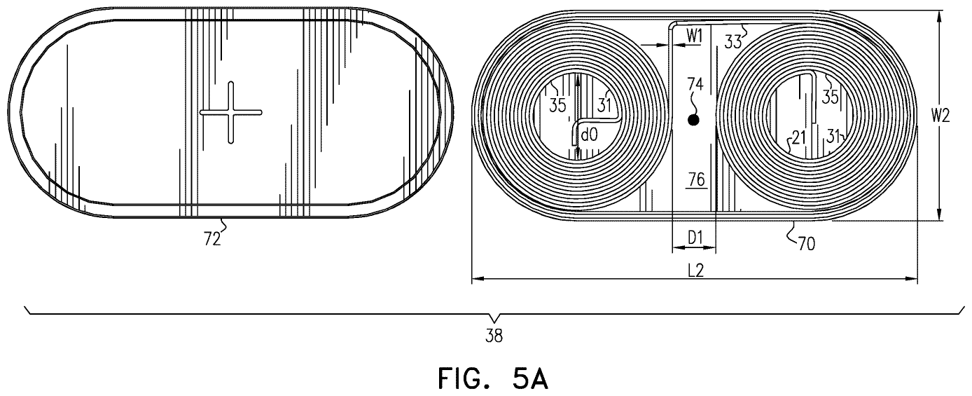

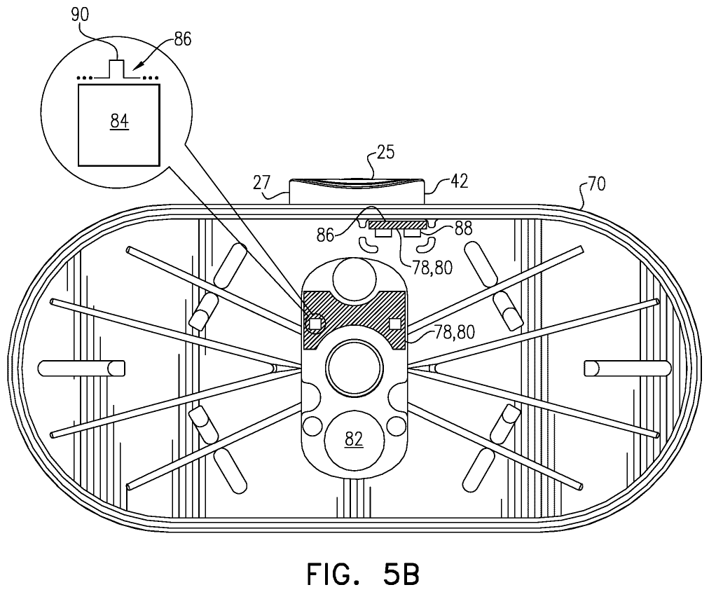

Typically, device 20 comprises a coil housing 38, which houses at least one stimulating coil 21 (FIG. 5A). Typically, coil housing 38 is attached to a handle 42.

In some embodiments, device 20 further comprises one or more straps 40 coupled to coil housing 38, e.g., via coupling knobs 49 on the coil housing, and configured to couple to cap 30 (FIG. 1), e.g., via a hook-and-loop fastener. As described above with reference to FIG. 1, magnetic-stimulation device 20 is typically connected to control unit 24 via cable 36. For example, cable 36 may comprise a first connecting interface 39, which is configured to connect to a complementary connecting interface 37 in the control unit, and—at the opposite end of the cable—a second connecting interface 41, which is configured to connect to a complementary connecting interface 43 in handle 42. In some embodiments, control unit 24 comprises a holder 45 configured to hold magnetic-stimulation device 20 when the device is not in use.

In some embodiments, device 20 further comprises a button 25, which may be located, for example, at the end of handle 42 opposite the end at which the handle connects to the cable. In some such embodiments, device 20 is configured to emit a test electromagnetic pulse in response to the pushing of button 25. The emission of test pulses may help in setting a patient-specific amplitude for the stimulating pulses.

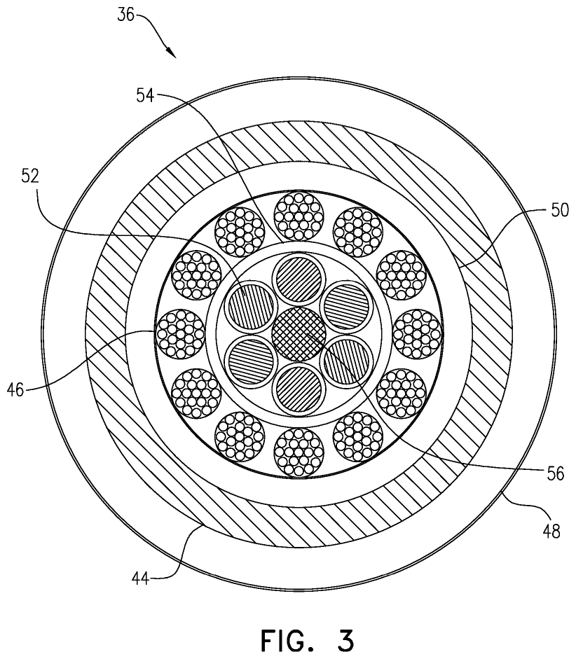

Reference is now made to FIG. 3, which is a schematic illustration of the cross-section of cable 36 indicated in FIG. 2B, in accordance with some embodiments of the present invention.

Typically, cable 36 is coaxial, comprising an inner conductor 46 and an outer conductor 44, which is coaxial with inner conductor 46. Typically, inner conductor 46 carries current to the magnetic-stimulation device from the control unit, while outer conductor 44 carries current to the control unit from the magnetic-stimulation device. An outer jacket 48 insulates outer conductor 44 from the surrounding environment (e.g., from the patient and clinician), while an inner jacket 50 insulates the two conductors from one another.

In some embodiments, cable 36 further comprises one or more insulated control wires 52, which pass through inner conductor 46 (e.g., at the center of cable 36), with another inner jacket 54 insulating the control wires from inner conductor 46. For example, FIG. 3 shows six control wires 52 surrounding a filler 56 at the center of the cable.

In some embodiments, magnetic-stimulation device 20 further comprises one or more temperature sensors, which may be contained, for example, within coil housing 38, as described below with reference to FIG. 5B. In such embodiments, typically, control wires 52 comprise wires for powering the temperature sensors and for serial communication with the temperature sensors. Alternatively or additionally, as described below with reference to FIG. 5B, the magnetic-stimulation device comprises electrically-activated status indicators (e.g., light emitting diodes (LEDs)), and control wires 52 include wires connected to these indicators.

Advantageously, in addition to providing electrical insulation, outer jacket 48 and inner jackets 50 and 54 provide structural stability, in that they hold the conducting elements of cable 36 in place. Nevertheless, cable 36 is sufficiently small, light, and flexible such that magnetic-stimulation device 20 (FIGS. 2A-B) may be worn comfortably. For example, outer jacket 48 (and hence, the cable) may have an outer diameter less than 1.4 cm (e.g., between 0.6 and 1.4 cm). Alternatively or additionally, the mass of cable 36 may be less than 700 g (e.g., between 300 and 700 g). Alternatively or additionally, the bend radius of cable 36 may be less than five times (e.g., between one and five times) the outer diameter of the cable.

Reference is now made to FIG. 4, which is a schematic illustration of cap 30, in accordance with some embodiments of the present invention.

Cap 30 comprises a piece 58 of material, which, in some embodiments, is framed by an elastic frame 62. Typically, to facilitate a better fit, the material is stretchable; for example, the material may comprise synthetic rubber and/or spandex. In some embodiments, piece 58 comprises multiple smaller pieces, which are joined (e.g., stitched) together during the manufacturing of the cap.

Cap 30 further comprises multiple coupling pads 64 coupled to piece 58 of material. Each of straps 40 (FIG. 2A) is configured to couple to any one of coupling pads 64 so as to couple the coil housing to the cap. For example, coupling pads 64 may comprise loops and straps 40 may comprise hooks configured to couple to the loops, or coupling pads 64 may comprise hooks and straps 40 may comprise loops configured to couple to the hooks, such that the magnetic-stimulation device is coupled to the cap via a hook-and-loop fastener.

Typically, cap 30 is shaped to define multiple electrode-holding orifices 22 shaped to hold respective electrodes 61, which are configured to record the electrical response of the brain to the magnetic stimulation. Typically, electrodes 61 are held within electrode-holding orifices 22 such that, when cap 30 is worn by the patient, the electrodes contact the patient's head.

Advantageously, coupling pads 64 are distributed across the surface of cap 30 such that magnetic-stimulation device 20 may be coupled to the cap at various locations, and hence, various regions of the patient's brain may be stimulated. Moreover, at each location, the coupling pads guide the placement of the magnetic-stimulation device, thereby facilitating a more effective stimulation. Typically, for greater stability, magnetic-stimulation device 20 comprises multiple (e.g., four) straps, and each of the straps is coupled to a different respective coupling pad.

For example, the coupling pads may be positioned on piece 58 of material such that, when the cap is worn on the head of the patient, the coil housing is couplable to the cap over the right or left primary motor cortex or dorsolateral prefrontal cortex of the patient's brain.

Typically, cap 30 further comprises multiple buckles 66 configured to couple to a chin strap, which helps to secure the cap on the patient's head.

Typically, cap 30 is further shaped to define multiple access orifices 68, via which the patient's head may be accessed. Thus, for example, an impedance-reducing gel, which reduces the impedance seen by the electrodes, may be applied to the patient's head via access orifices 68.

Reference is now made to FIG. 5A, which is a schematic illustration of coil housing 38, in accordance with some applications of the present invention.

Coil housing 38 comprises a compartment 70, which houses coil 21, and a cover 72, which is configured to cover coil 21 such that the coil is fully encapsulated and, typically, hermetically sealed, by the coil housing. In some embodiments, the coil housing comprises a layer 76 of material, such as epoxy, and the coil is sandwiched between layer 76 and cover 72. Layer 76 thus facilitates the encapsulation and, typically, hermetic seal of the coil.

Typically, compartment 70 and cover 72 are made from a plastic. Further typically, during the manufacture of the coil housing, cover 72 is welded (e.g., ultrasonically welded) to compartment 70 after insertion of the coil.

During use, handle 42 (FIGS. 2A-B) is coupled to compartment 70 opposite cover 72 (i.e., at the opposite face of the compartment that is not shown in FIG. 5A), and coil housing 38 is placed over the patient's head such that cover 72 faces the head. For example, cover 72 may contact cap 30 (FIG. 4).

Typically, the magnetic-stimulation coil comprises a flattened wire 33, i.e., a wire shaped as a band or a strip. Wire 33, which is covered along its length by a thin electrically-isolating cover, is wound into two windings 31 in opposite directions, i.e., the wire is wound clockwise in one winding 31 and counterclockwise in the other winding. Although windings 31 may have any suitable shape (e.g., an elliptical shape or a square shape), windings 31 are typically circular (i.e., disk-shaped), and hence, are referred to herein as circles 35. Circles 35 are separated from each other (i.e., the outer edges of the circles are separated from one another at their greatest proximity to one another) by a distance D1 that is typically more than 10 mm (e.g., more than 12 mm) and/or less than 18 mm (e.g., less than 16 mm), for example, 10 mm 18 mm or 12 mm-16 mm.

An advantage of the two circles is greater localization of the magnetic field, relative to a single loop of wire. In particular, the two circles destructively interfere with one another toward the edges of the coil and constructively interfere near the middle of the coil (particularly between the two circles), such that the magnetic field is concentrated near the center of the coil. Distance D1 affects the distance from the coil at which the magnetic field is concentrated; in particular, the latter distance is an increasing function of D1. In general, it is desired that the magnetic field be concentrated at least 2 cm beneath the patient's scalp with minimal near-field effects, which might cause discomfort to the patient. The example values for D1 provided above typically satisfy this objective.

For some applications, the width W1 of the flattened wire is more than 4 mm (e.g., more than 5 mm) and/or less than 7 mm (e.g., less than 6 mm), for example, 4 mm-7 mm, or 5 mm-6 mm. Alternatively or additionally, the thickness of the flattened wire (i.e., the dimension of the wire that goes into the page in FIG. 5A) is more than 0.8 mm (e.g., more than 1 mm) and/or less than 1.6 mm (e.g., less than 1.4 mm), for example, 0.8 mm-1.6 mm, or 1 mm-1.4 mm. Alternatively or additionally, the ratio between the width of the flattened wire and the thickness of the flattened wire is more than 3:1 (e.g., more than 4:1) and/or less than 6:1 (e.g., less than 5:1), for example between 3:1 and 6:1 or between 4:1 and 5:1.

In general, an advantage of a larger width and/or smaller thickness, relative to a smaller width and/or larger thickness, is that the current flowing through the coil is concentrated near the head of the patient, such that a sufficiently-strong magnetic field can be generated without overly increasing the size or mass of the coil. The example ranges described above provide this advantage without overly thinning or widening the wire.

For example, in some embodiments, by virtue of the small size of the coil, the length L2 of the housing is less than 150 mm, e.g., less than 140 mm, and/or the width W2 of the housing is less than 75 mm, e.g., less than 65 mm, and/or the thickness of the housing is less than 9 mm, e.g., less than 8 mm. Alternatively or additionally, by virtue of the small mass of the coil, the combined mass of the coil and housing is less than 500 g, e.g., less than 400 g.

It can be shown, via simulation, that if the inner diameter do of each of the circles is too small, the strength of the magnetic field is adversely affected. Hence, typically, the inner diameter do of each of the circles is between 21 and 31 mm, such as between 24 and 28 mm. This range of values for d0 facilitates a relatively small size of the coil without adversely affecting the strength of the magnetic field.

For some applications, each of the circles includes between 6 and 10 complete turns of the flattened wire, for example, 8 complete turns of the flattened wire. For other applications, each of the circles includes between 10 and 16 complete turns of the flattened wire, for example, 12-14 complete turns of the flattened wire. In general, a greater number of turns increases the strength of the magnetic field (provided that d0 remains large enough, as noted above).

By virtue of the small size and weight of the coil, the magnetic-stimulation device can be readily positioned (and repositioned, if required) in close proximity to cap 30 (FIG. 4), e.g., directly on the cap, without causing discomfort to the patient. In contrast, a larger or heavier coil would cause discomfort or require the support of a stand. As described above with reference to FIG. 4, for some applications, the magnetic-stimulation device is reversibly coupled to the cap, for example, using hook and loop fasteners. Typically, this maintains the position of the magnetic-stimulation device relative to the patient's head even if the patient moves their head.

As described above with reference to FIG. 1, typically, magnetic-stimulation coil 21 is used in a diagnostic procedure, as opposed to a transcranial magnetic stimulation (TMS) treatment procedure. As further described above with reference to FIGS. 1 and 4, system 28 comprises, in addition to magnetic-stimulation device 20, a plurality of electrodes 61, which are typically arranged within cap 30, which is placed on the patient's head. System 28 further comprises at least one computer processor 32. To perform the diagnostic procedure, magnetic-stimulation device 20 is reversibly coupled to cap 30, e.g., as described above with reference to FIG. 4. Subsequently, processor 32 drives the magnetic-stimulation device to apply magnetic stimulation to the patient's brain via the magnetic-stimulation coil, receives a magnetic-stimulation-evoked signal that is detected by the electrodes, and diagnoses the patient by analyzing the magnetic-stimulation-evoked signal.

Typically, processor 32 drives the magnetic-stimulation device to apply the magnetic stimulation to a depth of at least 2 cm from the patient's scalp. In other words, the processor provides coil 21 with power that, given the parameters of the coil described herein, is sufficient to cause the magnetic field to penetrate to this depth at a strength that is sufficient for stimulating the patient's brain. Advantageously, by virtue of the parameters of the coil, the stimulation is applied to this depth without the coil overheating and without the coil causing discomfort by heating the scalp of the patient.

For some applications, the frequency of the magnetic stimulation within each pulse is between 2 kHz and 4 kHz, e.g., between 2.5 kHz and 3.5 kHz. Alternatively or additionally, the length of each pulse is between 200 microseconds and 400 microseconds, e.g., between 250 microseconds and 350 microseconds. Alternatively or additionally, the magnetic stimulation is applied at an amplitude of between 4000 A and 6000 A, e.g., between 4500 A and 5500 A.

Reference is now made to FIG. 5B, which is a schematic illustration of compartment 70, in accordance with some applications of the present invention. The view of compartment 70 shown in FIG. 5B corresponds to that of FIG. 5A, with coil 21 and layer 76 hidden from view.

In some embodiments, the magnetic-stimulation device comprises at least one printed circuit board (PCB) 78 comprising circuitry 80 configured to produce an output for facilitating the stimulation procedure. Each PCB 78 comprises at least one filtering wire 86 configured to protect the circuitry 80 by filtering a common-mode voltage from coil 21, such that the coil housing may house the coil and the PCB without the common-mode voltage damaging the circuitry. For example, each PCB may be contained within compartment 70.

In some embodiments, circuitry 80 comprises one or more LEDs 84 configured to emit light indicating the stage of the stimulation procedure. Typically, the LEDs are controlled via control signals that pass through cable 36 from control unit 24 (FIG. 1), e.g., as described above with reference to FIG. 3. As the stage of the procedure changes (e.g., as the magnetic stimulation begins or ends), the control signals cause the LEDs to go on or off. In some embodiments, a different respective filtering wire 86 at least partly surrounds each LED.

Typically, the light from the LEDs passes through a light guide 27, which runs from the LEDs to the exterior of the coil housing such that the user can ascertain the state of the device from light guide 27. Typically, handle 42 comprises the exposed (and visible) end of light guide 27; for example, the exposed end of the light guide may surround button 25. (This embodiment is also shown in FIG. 2B.)

Alternatively or additionally, circuitry 80 comprises one or more temperature sensors 88 configured to output signals indicating the temperature within the coil housing. (For embodiments in which the device also comprises LEDs 84, temperature sensors 88 and LEDs 84 are typically disposed on different respective PCBs.) Typically, the signals are communicated to control unit 24 via cable 36 (FIG. 1), e.g., as described above with reference to FIG. 3. In some embodiments, filtering wire 86 runs along the face of the PCB (e.g., near the perimeter of the PCB) that is opposite the face on which temperature sensors 88 are disposed.

Typically, filtering wire 86 is floating, i.e., is not connected to any voltage source. Further typically, filtering wire 86 is shaped to define a series of rectangular waves 90, whose properties determine the properties of the filter.

Typically, one or more openings 82 in compartment 70 facilitate the passage of wires connected to button 25, coil 21, the PCBs, and the cable, and/or facilitate airflow.

It is noted that the embodiments described with reference to FIG. 5B may be combined with any suitable coil configured to magnetically stimulate the brain of a patient during a stimulation procedure for diagnostic or treatment purposes.

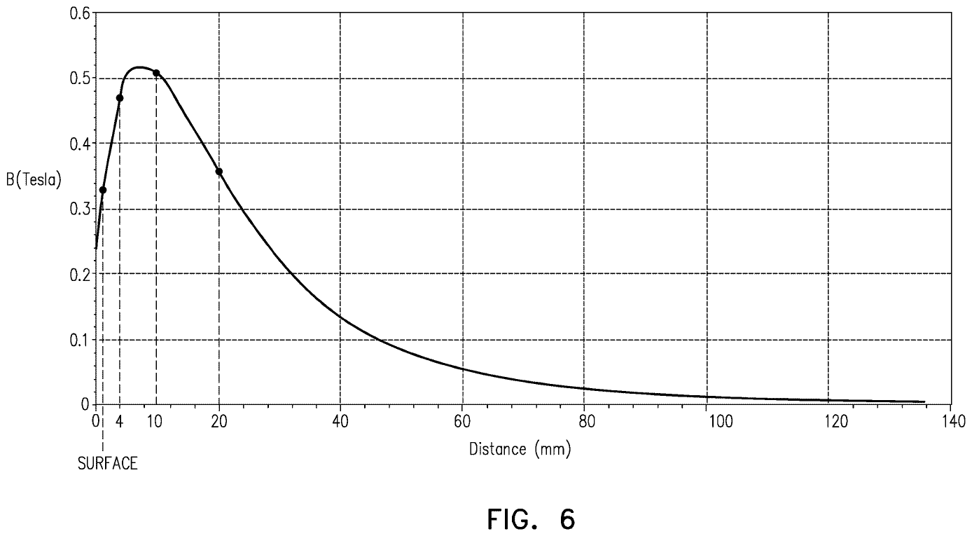

Reference is now made to FIG. 6, which shows results of a simulated magnetic stimulation using a coil having parameters in accordance with some embodiments of the present invention. FIG. 6 shows the simulated flux density (B) at various distances from the surface of the coil that, in an actual stimulation procedure, would face the head of the patient. The flux density is measured along a central axis 74 of the coil, which, as shown in FIG. 5A, runs perpendicularly to compartment 70. The word “surface” as marked in FIG. 6 refers to the outer surface of the housing (e.g., the outer surface of cover 72), whereas the distances indicated along the x-axis are measured from the surface of the coil itself. The outer surface of the housing is typically within 2 mm, e.g., within 1 mm, of the surface of the coil; in an actual stimulation procedure, this surface would contact the head of the patient.

In some embodiments, the magnetic-stimulation coil is configured to generate a magnetic field having a flux density (B) of less than 0.5 Tesla (e.g., less than 0.4 or 0.3 Tesla) at the coil surface (corresponding to a distance of zero in FIG. 6), and/or a flux density of less than 0.5 Tesla (e.g., less than 0.4 or 0.35 Tesla) at the surface of the housing. Nonetheless, the flux density is typically greater than 0.3 Tesla (e.g., greater than 0.35 Tesla) at a 2 cm distance along central axis 74. Thus, the magnetic field is sufficiently strong for stimulation at 2 cm, yet is not too strong at the patient's scalp. One reason for this is that, by virtue of the properties of the coil described herein, the flux density changes relatively slowly along central axis 74. For example, as shown in FIG. 6, the flux density may reach a maximum at a distance of more than 4 mm, e.g., more than 6 mm or 7 mm, from the surface of the coil, before gradually decreasing. As a result, the flux density is greatest within the brain (where stimulation is required), and is relatively high even at 2 cm. In contrast, for other coils (i) the flux density might be greatest outside the brain, and/or (ii) the flux density would need to be uncomfortably high at the patient's scalp in order to be sufficiently high at 2 cm.

The advantageous feature of the magnetic field described above may be expressed as a ratio. For example, in some embodiments, the ratio of the magnetic-field strength (which is linearly related to the flux density and is conventionally indicated by “H”) at a 2 cm distance from the surface of the coil along central axis 74 to the magnetic-field strength at the coil surface is greater than 2:3, e.g., greater than 1:1 or 1:0.9.

Alternatively or additionally, the magnetic-stimulation coil generates dB/dt of less than 8e⁻³ (e.g., less than 7e⁻³) Tesla/microsecond at the coil surface and dB/dt of more than 7e⁻³ (e.g., more than 7.5e⁻³) Tesla/microsecond at a 2 cm distance along central axis 74. Alternatively or additionally, the ratio of dB/dt at 2 cm to dB/dt at the coil surface is greater than 2:3, e.g., greater than 1:1 or 1:0.9.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 32. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor (e.g., computer processor 32) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

Computer processor 32 is typically a hardware device programmed with computer program instructions to produce a special purpose computer. For example, when programmed to perform the algorithms described with reference to the figures, computer processor 32 typically acts as a special purpose diagnostics computer processor. Typically, the operations described herein that are performed by computer processor 32 transform the physical state of a memory, which is a real physical article, to have a different magnetic polarity, electrical charge, or the like depending on the technology of the memory that is used. For some applications, operations that are described as being performed by computer processor 32 are performed by a plurality of computer processors in combination with each other.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus, comprising:
a magnetic-stimulation device comprising:
a magnetic-stimulation coil that is formed from a flattened wire that is wound such as to form two circles that are separated from each other, a ratio between a width and a thickness of the flattened wire being greater than 3:1; and
a housing that houses the magnetic-stimulation coil.

2. The apparatus according to claim 1, wherein an inner diameter of each of the circles is between 21 and 31 mm.

3. The apparatus according to claim 2, wherein the inner diameter is between 24 and 28 mm.

4. The apparatus according to claim 1, wherein the flattened wire is wound such that each of the two circles includes between 6 and 10 complete turns of the flattened wire.

5. The apparatus according to claim 1, wherein the flattened wire is wound such that each of the two circles includes between 10 and 16 complete turns of the flattened wire.

6. The apparatus according to claim 1, wherein the housing hermetically seals the magnetic-stimulation coil.

7. The apparatus according to claim 1, wherein the housing comprises a compartment and a cover that are welded to each other.

8. The apparatus according to claim 1, wherein the ratio between the width and the thickness of the flattened wire is between 3:1 and 6:1.

9. (canceled)

10. The apparatus according to claim 1, wherein the width of the flattened wire is more than 4 mm.

11. (canceled)

12. The apparatus according to claim 1, wherein the thickness of the flattened wire is less than 1.6 mm.

13. (canceled)

14. The apparatus according to claim 1, wherein the two circles are separated from each other by more than 10 mm.

15-16. (canceled)

17. The apparatus according to claim 1,
wherein the magnetic-stimulation coil is configured to generate a magnetic field having a magnetic-field strength, and
wherein a ratio of the magnetic-field strength at a 2 cm distance from a surface of the coil along a central axis of the coil to the magnetic-field strength at the surface of the coil is greater than 2:3.

18. (canceled)

19. A system, comprising:
the apparatus according to claim 1;
a plurality of electrodes; and
at least one computer processor configured to:
drive the magnetic-stimulation device to apply a magnetic stimulation to a brain of the patient via the magnetic-stimulation coil,
receive a magnetic-stimulation-evoked signal that is detected by the electrodes, and
diagnose the patient, by analyzing the magnetic-stimulation-evoked signal.

20. The system according to claim 19, wherein the at least one computer processor is configured to drive the magnetic-stimulation device to apply the magnetic stimulation to a depth of at least 2 cm from a scalp of the patient.

21. The system according to claim 20, wherein the magnetic-stimulation coil is configured not to overheat when the magnetic stimulation is applied to the depth of at least 2 cm.

22. The system according to claim 20, wherein the magnetic-stimulation coil is configured not to cause discomfort by heating a scalp of the patient when the magnetic stimulation is applied to the depth of at least 2 cm.

23. The system according to claim 19, wherein the plurality of electrodes are arranged within a cap that is configured to be placed on a head of the patient.

24. The system according to claim 23, wherein the magnetic-stimulation device is configured to be reversibly coupled to the cap.

25-29. (canceled)

30. A system, comprising:
a cap configured for wearing on a head of a patient, the cap comprising:
a piece of material; and
multiple coupling pads coupled to the piece of material; and
a magnetic-stimulation device, comprising:
a coil housing;
one or more straps coupled to the coil housing, each of the straps being configured to couple to any one of the coupling pads so as to couple the coil housing to the cap while the cap is worn on the head of the patient; and
a coil housed within the coil housing and configured to magnetically stimulate a brain of the patient while the coil housing is coupled to the cap.

31-35. (canceled)

36. The system according to claim 30, wherein a mass of the coil housing, with the coil, is less than 500 g.

37-47. (canceled)
